(19) United States
(12) Patent Application Publication
(10) Pub. No.: US 2008/0228112 A1
(43) Pub. Date: Sep. 18, 2008

(54) Shock wave conductor

(76) Inventor: Axel Voss, Kreuzlingen (CH)

Correspondence Address: Raymond R. Ferrera, 1221 McKinney Street, Suite 4400, Houston, TX, 77010, US

(21) Appl. No.: 11/807,404

(22) Filed: May 29, 2007

Related U.S. Application Data
(60) Provisional application No. 60/803,985, filed on Jun. 6, 2006.

(30) Foreign Application Priority Data
Jun. 6, 2006 (DE) 10 2006 026 232.8

(51) Int. Cl. A61B 17/225; A61H 23/00; A61N 7/00

(52) U.S. Cl. 601/4

(57) ABSTRACT

The invention relates to a device for reducing the coupling-in surface area of a shock wave reflector and for forwarding a shock wave focus zone.

The invention relates to a device for reducing the coupling-in surface area of a shock wave reflector and for forwarding a shock wave focus zone.

Shock waves are used in numerous fields of medicine.

The most widely known field comprises the therapeutic and cosmetic use in the treatment of e.g. stone-type disorders (for example urolithasis, cholelithasis) and scar treatment in human and animal medicine.

More recent fields of use include dental treatment, the treatment of arthrosis, the removal of calcium deposits (e.g. Tendinosis calcarea), the treatment of chronic tennis elbow or golfer's elbow (known as Epicondylopathia radialis or ulnaris), the treatment of chronic shoulder pain (known as rotator cuff tendinosis) and of chronic tears of the Achilles tendon (known as achillodynia).

Furthermore, shock waves are also used for therapy in the case of osteoporosis, paradontosis, non-healing bone fractures (known as pseudoarthrosis), bone necrosis and other disorders. More recent studies are also examining use in stem cell therapies.

Shock waves can also be used to exert mechanical stress, e.g. in the form of shear forces, on cells, with apoptosis thereof being induced. This takes place for example by initiating the “death receptor pathway” and/or the cytochrome c pathway and/or a caspase cascade.

Apoptosis is understood to mean the initiation of a genetically controlled program which leads to the “cell suicide” of individual cells in the tissue. In the process, the respective cells and their organelles shrivel up and break into pieces known as apoptotic bodies. These are then phagocytozed by macrophages and/or neighboring cells. Apoptosis is therefore a non-necrotic cell death without an inflammation reaction.

The use of shock waves is therefore advantageous in all cases involving treatment of disorders with a reduced rate of apoptosis, e.g. tumor treatments or viral diseases.

Shock waves can also be used with particular advantage to treat necrotically changed areas and structures in the muscular tissue, in particular in the myocardium, to induce the build-up of cartilage in arthritic joint disorders, to initiate the differentiation of embryonic or adult stem cells in vivo and in vitro according to the surrounding cell layers, to treat tissue defects, in particular cellulitis, and to break down fat cells and also to activate growth factors, in particular TGF-[beta].

Shock waves can also be used to prevent oedema formation and/or spread and also for oedema breakdown, for the treatment of ischemia, rheumatism, joint disorders, jaw bones (paradontitis), cardiological disorders and cardial infarction, paresis (paralysis), neuritis, paraplegia, arthrosis, arthritis, for the prophylaxis of keloids, for the treatment of keloids and neuromas, for the treatment of achillobursitis and other types of bone necrosis.

Another use relates to the treatment of spinal cord and nerve injuries, for example spinal cord injuries with associated oedema formation.

Shock waves are also suitable for treating scarred tendon and ligament tissue and also open wounds which do not heal easily.

Such open wounds and sores which do not heal easily are known as ulcers or ulceration. They represent surface damage caused by a breakdown of tissue on the skin and/or mucous membrane. Depending on which tissue areas are affected, in the case of surface lesions these are referred to as exfoliation (only the epidermis is affected) or excoriation (both the epidermis and the dermis is affected).

Open wounds which can be treated with shock waves include in particular ulcus cruris, ulcus hypertonicum, ulcus varicosum or ulcus terebrans due to an improved healing process brought about thereby.

Shock waves are also suitable for stimulating cell reproduction and the differentiation of stem cells.

Essentially three principles are available for generating shock waves, namely the electrohydraulic, the piezoelectric and the electromagnetic principle.

In the case of an electrohydraulic shock wave generator, in the focus of a reflector a spark discharge is produced between two electrode tips in a liquid medium. As soon as the expansion speed of the plasma bubble produced by the spark discharge has dropped below the sound speed, a shock wave detaches from the surface of the plasma bubble and propagates as the primary, divergent shock wave. A significant part of the divergent wave is reflected by the reflector.

The reflector here may be of parabolic or elliptical shape.

If the reflector is of elliptical shape, the reflected part of the divergent shock wave is transformed by the reflection into a convergent shock wave which converges in a second focus and then diverges. If the position of the second focus is to be changed, an acoustic lens must be used.

If the reflector is of parabolic shape, the reflected part of the divergent shock wave is transformed by the reflection into a planar shock wave which runs in a parallel manner from the reflector. In order to produce another focus in the field of application, an acoustic lens which focuses the shock wave is required.

Systems for treatment using shock waves according to the prior art are oriented towards using a treatment head to couple the shock waves into a body via a coupling pad attached to the treatment head.

However, on account of their dimensions, the treatment heads according to the prior art cannot be used for coupling the shock waves locally and over a small surface area into a body.

With treatment heads according to the prior art, therefore, it is not possible to reach many areas of a body directly, and the shock waves have to be passed through areas of the body located in front of the actual treatment area.

This may lead to undesirable side effects such as the occurrence of haematomas for example, or the effectiveness of the shock waves may be reduced due to reflection at tissue boundary layers. This occurs when the shock waves have to pass through adjacent areas with a very different structure on their way through the body.

It would therefore be desirable if the shock waves could be coupled directly into the area to be treated.

It is therefore an object of the present invention to provide a device which makes it possible to couple shock waves locally and over a small surface area into an area to be treated.

This object is achieved by a shock wave reflector with a shock wave conductor according to the invention.

A shock wave reflector with a shock wave conductor according to the invention includes a reflector and a shock wave conductor.

The shock wave conductor includes an acoustic funnel and a shock wave tube adjoining the acoustic funnel.

The acoustic funnel serves to reduce the cross-sectional dimensions of the shock wave reflector with a shock wave conductor according to the invention perpendicular to a shock wave coupling-in direction, so that the cross-sectional area of the shock wave tube is much smaller than that of the reflector.

In this case, the shock waves coming from the reflector are converged in the acoustic funnel and conveyed into the shock wave tube adjoining the acoustic funnel.

The acoustic funnel preferably has a conical shape, but non-conical outer surfaces which lead to a reduction in the cross-sectional dimensions are also possible.

Furthermore, the acoustic funnel is designed in such a way that a second virtual focus of the reflector is located directly at the transition from the acoustic funnel into the shock wave tube, and thus the focus is successfully reproduced in the shock wave tube.

The acoustic funnel may be combined in one part with the reflector or may form a separate part which is connected to the reflector by means of screws for example.

In the case of a separate acoustic funnel, the reflector and the acoustic funnel may have separating walls in the region of the common contact area, so that the acoustic funnel together with the shock wave tube can easily be separated from a first reflector and connected to a second reflector.

In this case, the acoustic funnel can be connected to a treatment head according to the prior art and used instead of a coupling pad.

The shock wave tube serves to provide a surface for coupling the shock waves into a body and to bridge over a distance by passing the shock Waves through the shock wave tube from a first end, which is connected to the acoustic funnel, to a second end.

The second end of the shock wave tube has a surface for coupling the shock waves into a body. The surface is preferably arranged perpendicular to the longitudinal axis of the shock wave tube and is preferably much smaller than a coupling-in surface of a shock wave reflector according to the prior art, so that the shock waves can be coupled into the body over a small surface area by means of a shock wave reflector with a shock wave conductor according to the invention.

The transition from the acoustic funnel into the shock wave tube is preferably designed in such a way that the radius in the wall is 1000-10,000 times the wavelength of the shock waves (approx. 10⁻⁶ m). Undesirable reflections of the shock waves due to a kink in the wall are thus reduced.

The shock wave tube of the shock wave conductor preferably has a narrow, elongate shape.

Furthermore, the shock wave tube is preferably designed to be flexible so that it can be passed for example through body openings or lumens which are not straight.

The inner diameter of the shock wave tube is selected in such a way that the shock wave tube completely surrounds a −6 dB focus zone (area of the shock wave field in which the local pressure is greater than or equal to half the maximum pressure).

The inner diameter of the shock wave tube is thus preferably 1 mm to 5 mm.

The outer diameter of the shock wave tube is preferably 1.0 mm to 10 mm.

The length of the shock wave tube is preferably 1 cm to 500 cm.

Located in the interior of the shock wave conductor is a medium which has a similar acoustic impedance and a similar sound speed to water and which conducts the shock waves from the reflector to the second end of the shock wave tube.

The medium in the shock wave conductor preferably has a sound speed of 1300 m/s to 1800 m/s.

The reflection medium of the wall of the shock wave conductor has an acoustic impedance and sound speed very different to that of water and preferably consists of a metal or ceramic material.

The reflection medium of the wall of the shock wave conductor preferably has a sound speed of either less than 1300 m/s or more than 1800 m/s.

In one preferred embodiment, the shock wave reflector with a shock wave conductor according to the invention is sheathed in a sound-absorbing medium which reduces the primary sound wave in the audible range, thereby resulting in a more pleasant application for a person to be treated.

Due to its preferably elongate shape and its preferably small diameter in the region of the shock wave tube and also the flexibility of the shock wave tube, it is possible with the shock wave reflector with a shock wave conductor according to the invention to directly reach areas within a body through an opening in the body or through a minimally invasive percutaneous access, without having to pass the shock waves through other areas of the body located in front of the treatment area.

Furthermore, with the shock wave reflector with a shock wave conductor according to the invention, it is possible to couple shock waves locally and over a small surface area into an area to be treated.

The shock wave reflector with a shock wave conductor according to the invention thus opens up new fields of use for treatment with shock waves.

In fields of use that are already established, the shock wave reflector with a shock wave conductor according to the invention makes it possible to reduce the side effects of the treatment and to increase the effectiveness of the shock waves.

By way of example, possible fields of use which may be mentioned here include indications of the dental area and of the joints, tumors, and also coronary, cerebral-neurological and spinal-neurological indications.

The invention will be explained in more detail below with reference to the drawing.

In the drawing:

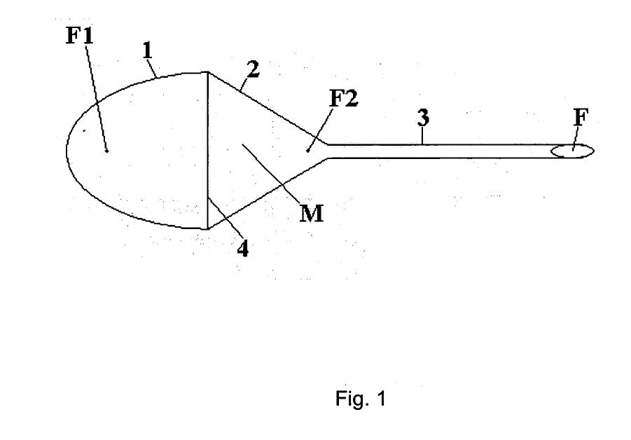

FIG. 1 shows a schematic view of a shock wave reflector with a shock wave conductor according to the invention.

A schematic view of one embodiment of a shock wave reflector with a shock wave conductor according to the invention can be seen in FIG. 1.

In the illustrated embodiment of the invention, in a first focus (F1) of a reflector (1) of a shock wave reflector with a shock wave conductor according to the invention a spark discharge is produced between two electrode tips in a liquid medium (M) with similar properties to water. A shock wave which detaches from the surface of a plasma bubble produced by the spark discharge is reflected by means of a suitable reflection medium of a wall of the reflector (1), which is preferably a material with an acoustic impedance greatly differing from water, and is bundled at a second virtual focus (F2) of the reflector (1).

A shock wave conductor is connected to the opening side of the reflector (1).

The shock wave conductor, which comprises an acoustic funnel (2) and a shock wave tube (3), is attached with the larger opening of the acoustic funnel (2) flush against the reflector (1).

The acoustic funnel (2) converges the shock waves coming from the reflector (1) and conveys them into the adjoining shock wave tube (3).

To this end, the acoustic funnel (2) is designed in such a way that the second virtual focus (F2) of the reflector (1) is located directly at the transition from the acoustic funnel (2) into the shock wave tube (3), and thus the focus is successfully reproduced in the shock wave tube (3).

In the illustrated embodiment, both the reflector (1) and the acoustic funnel (2) have a separating wall (4) in the region of the common contact area. It is thus easily possible to separate the acoustic funnel (2) together with the shock wave tube (3) from the reflector (1), in order for example to connect the acoustic funnel (2) together with the shock wave tube (3) to a second reflector.

In this case, the connection of the acoustic funnel (2) to a reflector (1) may be produced by means of screws.

The shock wave tube (3) at a first end tightly adjoins a smaller opening of the acoustic funnel (2) and is arranged on the side of the acoustic funnel (2) opposite the reflector (1).

In the shock wave tube (3), the shock waves are conveyed from the first end of the shock wave tube (3) to a second end of the shock wave tube (3).

The shock wave tube (3) is of circular-cylindrical shape and has an inner diameter which is selected in such a way that the shock wave tube (3) completely surrounds a −6 dB focus zone (F).

The second end of the shock wave tube (3) is closed and serves to couple the shock waves into an area to be treated, which area is preferably located within a body and can be reached by the shock wave reflector with a shock wave conductor according to the invention via an opening in the body or via a minimally invasive percutaneous access.

The interior of the shock wave conductor preferably contains the same medium (M) as in the reflector (1), which medium has a similar acoustic impedance and a similar sound speed to water.

In order to be able to reach the area, which is preferably located within a body, with the second end of the shock wave tube (3) via an opening in the body or via a minimally invasive percutaneous access using the shock wave reflector with a shock wave conductor according to the invention, the shock wave tube (3) preferably has a narrow and elongate shape and is preferably designed to be flexible so that the shock wave tube (3) can be passed through the opening in the body or through the minimally invasive percutaneous access to the area within the body.

The shock wave reflector with a shock wave conductor according to the invention thus opens up new fields of treatment for shock wave therapy. Mention may be made in particular of treatments using shock waves in the dental area and also minimally invasive applications of shock waves in the interior of the body, such as coronary applications for example.

List of references

| | | |
|---|---|---|
| 1 | F | focus zone |
| 2 | F1 | first focus of the reflector |
| 3 | F2 | second virtual focus of the reflector |
| 4 | M | medium in the shock wave conductor |
| 5 | 1 | reflector |
| 6 | 2 | acoustic funnel |
| 7 | 3 | shock wave tube |
| 8 | 4 | separating wall |

1. A shock wave reflector with a shock wave conductor, including a reflector, an acoustic funnel for reducing the cross-sectional dimensions of the shock wave conductor perpendicular to a shock wave coupling-in direction, and a shock wave tube for conducting shock waves from a first end to a second end and for coupling-in the shock waves.

2. The shock wave reflector with a shock wave conductor according to claim 1, wherein the acoustic funnel is designed in such a way that a second virtual focus of the reflector is located directly at the transition from the acoustic funnel into the shock wave tube.

3. The shock wave reflector with a shock wave conductor according to claim 1, wherein the acoustic funnel has a conical shape.

4. The shock wave reflector with a shock wave conductor according to claim 1, wherein a separating wall is present between the reflector and the acoustic funnel.

5. The shock wave reflector with a shock wave conductor according to claim 1, wherein the reflector and the acoustic funnel are releasably connected.

6. The shock wave reflector with a shock wave conductor according to claim 5, wherein the connection of the reflector to the acoustic funnel is produced by means of screws.

7. The shock wave reflector with a shock wave conductor according to claim 1, wherein the reflector is a treatment head for the coupling-in of shock waves.

8. The shock wave reflector with a shock wave conductor according to claim 1, wherein the transition from the acoustic funnel into the shock wave tube is designed in such a way that a radius in the wall is 1000-10,000 times the wavelength of the shock waves.

9. The shock wave reflector with a shock wave conductor according to claim 1, wherein the shock wave conductor includes a medium for conducting the shock waves, the sound speed of which medium lies in a range from 1300 m/s to 1800 m/s.

10. The shock wave reflector with a shock wave conductor according to claim 1, wherein the wall of the shock wave conductor includes a reflection medium which consists of a metal material.

11. The shock wave reflector with a shock wave conductor according to claim 1, wherein the wall of the shock wave conductor includes a reflection medium which consists of a ceramic material.

12. The shock wave reflector with a shock wave conductor according to claim 1, wherein the wall of the shock wave conductor includes a reflection medium, the sound speed of which is less than 1300 m/s.

13. The shock wave reflector with a shock wave conductor according to claim 1, wherein the wall of the shock wave conductor includes a reflection medium, the sound speed of which is more than 1800 m/s.

14. The shock wave reflector with a shock wave conductor according to claim 1, wherein the shock wave tube is flexible.

15. The shock wave reflector with a shock wave conductor according to claim 1, wherein the shock wave tube has an outer diameter in the range from 1.0 mm to 10 mm.

16. The shock wave reflector with a shock wave conductor according to claim 1, wherein the shock wave tube has an inner diameter which completely surrounds a −6 dB focus zone.

17. The shock wave reflector with a shock wave conductor according to claim 1, wherein the shock wave tube has an inner diameter in the range from 1 mm to 5 mm.

18. The shock wave reflector with a shock wave conductor according to claim 1, wherein the shock wave tube has a length in the range from 1 cm to 500 cm.

19. The shock wave reflector with a shock wave conductor according to claim 1, wherein the shock wave reflector with a shock wave conductor is sheathed in a sound-absorbing medium.

20. The use of a shock wave reflector with a shock wave conductor according to claim 1 for treating an indication of the dental area with shock waves.

21. The use of a shock wave reflector with a shock wave conductor according to claim 1 for treating an indication of the joints with shock waves.

22. The use of a shock wave reflector with a shock wave conductor according to claim 1 for treating tumors with shock waves.

23. The use of a shock wave reflector with a shock wave conductor according to claim 1 for treating coronary indications with shock waves.

24. The use of a shock wave reflector with a shock wave conductor according to claim 1 for treating cerebral-neurological indications with shock waves.

25. The use of a shock wave reflector with a shock wave conductor according to claim 1 for treating spinal-neurological indications with shock waves.
